(19) United States
(12) Patent Application Publication
(10) Pub. No.: US 2009/0216267 A1
(43) Pub. Date: Aug. 27, 2009

(54) CLOSURE DEVICE WITH RAPIDLY DISSOLVING ANCHOR

(75) Inventors: Martin R. Willard, Burnsville, MN (US); Dave Sogard, Edina, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/391,638

(22) Filed: Feb. 24, 2009

Related U.S. Application Data
(60) Provisional application No. 61/031,456, filed on Feb. 26, 2008.

(51) Int. Cl. A61B 17/08

(52) U.S. Cl. 606/213

(57) ABSTRACT

Closure devices with a rapidly dissolving anchor, systems delivering closure devices, and methods for making and using the same. An example closure device for closing an opening in a body lumen may include a plug, a rapidly dissolving anchor, and a suture coupling the plug to the anchor. The rapidly dissolving anchor may be configured to dissolve within the body lumen within about 30 days or less. At least a portion of the plug may be disposed adjacent an exterior surface of the body lumen. At least a portion of the rapidly dissolving anchor may be disposed within the body lumen.

### RELATED APPLICATIONS

This application claims priority to U.S. Provisional Application No. 61/031,456 filed Feb. 26, 2008.

### FIELD OF THE INVENTION

The present invention pertains to closure devices for closing an opening in a body lumen, systems for delivering closure devices, and methods for making and using the same. More particularly, the present invention pertains to closure devices having a rapidly dissolving anchor.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

The non-invasive medical procedures identified above typically gain access to the vasculature through an opening formed into the femoral artery. For obvious reasons, once the procedure is completed the opening the femoral artery will need to be closed. This may include applying direct pressure at the wound site. Alternatively, a device may be used to assist in the closing of the artery.

A wide variety of medical devices have been developed for medical use, for example, use in non-invasive medical procedures. Some of these devices include devices for closing an opening in a body lumen such as the femoral artery. Of the known medical devices, each has certain advantages and disadvantages. There is an ongoing need to provide alternative medical devices as well as alternative methods for manufacturing and using medical devices.

### BRIEF SUMMARY

The invention provides design, material, manufacturing method, and use alternatives for closure devices with a rapidly dissolving anchor and systems delivering closure devices. An example closure device for closing an opening in a body lumen may include a plug, a rapidly dissolving anchor, and a suture coupling the plug to the anchor. The rapidly dissolving anchor may be configured to dissolve within the body lumen within about 30 days or less. At least a portion of the plug may be disposed adjacent an exterior surface of the body lumen. At least a portion of the rapidly dissolving anchor may be disposed within the body lumen.

An example system for delivering a closure device to an opening in a body lumen to close the opening may include a sheath, a closure device disposed in the sheath, and a push member disposed in the sheath adjacent the closure device. The closure device may include a plug, a rapidly dissolving anchor, and a suture coupling the plug to the anchor. The rapidly dissolving anchor may be configured to dissolve within the body lumen within about 24 hours or less. The push member may be configured to advance the closure device out from the sheath.

An example method for closing an opening in a body lumen may include providing a closure device delivery system, disposing a portion of the sheath within the body lumen, advancing the closure device out from the sheath with the push member, closing the opening in the body lumen with the closure device, and removing the sheath and the push member from the body lumen. The system may include a sheath, a closure device disposed in the sheath, and a push member disposed in the sheath adjacent the closure device. The closure device may include a plug, a rapidly dissolving anchor, and a suture coupling the plug to the anchor. The rapidly dissolving anchor may be configured to dissolve within the body lumen within about 24 hours or less. The push member may be configured to advance the closure device out from the sheath.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is partial cross-section plan view of an example closure device disposed in and closing an opening in a body lumen;

FIG. 2 is a side view of an example anchor;

FIG. 3 is a cross-sectional view taken through line 3-3 in FIG. 2;

FIG. 4 is a cross-sectional view of another example anchor;

FIG. 5A is a cross-sectional view of another example anchor;

FIG. 5B is a cross-sectional view of another example anchor;

FIG. 6 is a cross-sectional view of another example anchor;

FIG. 7 is a cross-sectional view of another example anchor;

FIG. 8 is a cross-sectional view of another example anchor;

FIG. 9 is a side view of an example device for delivering a closure device to an opening in a body lumen;

FIG. 10 is a partial cross-sectional view of the delivery device shown in FIG. 9 delivering the closure device to the opening in the body lumen;

FIG. 11 is a partial cross-sectional view illustrating the deployment of the closure device in the body lumen;

FIG. 12 is a partial cross-sectional view further illustrating the deployment of the closure device in the body lumen;

FIG. 13 is a partial cross-sectional view of the closure device in the body lumen;

FIG. 14 is a partial cross-sectional view of the closure device in the body lumen where the anchor has dissolved; and

FIG. 15 is a perspective view of an alternative anchor.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein, the term “sugar” refers to carbohydrates including monosaccharides, disaccharides, oligosaccharides, and polysaccharides having, for example, four (tetrose), five (pentose), six (hexose), seven (heptose), or more carbon atoms. Some examples of monosaccharides sugars include allose, altrose, glucose, mannose, gulose, idose, galactose, talose, ribose, arabinose, xylose, lyxose, erthrose, threose, and glyceraldehyde. Some examples of disaccharides include cellobiose, maltose, lactose, gentiobiose, and sucrose. Some examples of oligosaccharides and/or polysaccharides include cellulose, starch, amylase, amylase, amylopectin, and glycogen. The sugar may be an aldose sugar (i.e., a sugar having an aldehyde functional group) or a ketose sugar (i.e., a sugar having a ketone functional group). The sugar may be a reducing sugar (i.e., a sugar oxidized by Tollens' reagent, Benedict's reagent, or Fehling's reagent) or a non-reducing sugar (i.e., a sugar not oxidized by Tollens' reagent, Benedict's reagent, or Fehling's reagent). The sugar may be cyclic (e.g., furanose, pyranose, etc.) or non-cyclic. The sugar may be either the D or L enantiomer, may rotate polarized light in either the (+) or the (−) direction, and may be either the α a or β anomer.

The following detailed description should be read with reference to the drawings in which similar elements in different drawings are numbered the same. The drawings, which are not necessarily to scale, depict illustrative embodiments and are not intended to limit the scope of the invention.

Less invasive cardiac diagnostic and/or treatment interventions (e.g., angioplasty) are an attractive and efficacious alternative to more invasive procedures such as cardiac bypass. These less invasive procedures typically involve a peripheral puncture that provides access to the vasculature (e.g., at the femoral vein). Once the proper access is obtained, the procedure can be carried out. Following the intravascular procedure, it may be necessary to take one or more steps to close the puncture. This may include application of direct pressure at the wound site. Alternatively, a closure device may be used to seal the opening in the vasculature.

A number of different closure devices exist for closing openings in a body lumen including openings into the femoral artery. These devices typically include an interior or intravascular component called an anchor or backstop, an exterior component called a plug (typically made from collagen), and a filament or suture connecting the anchor an plug together. The anchor is typically made from a relatively slow dissolving or absorbable polymer that is designed to dissolve over a period of about 60-90 days. A number of devices exist that have this general design and at least some of these devices are medically and commercially successful.

Notwithstanding the medical and commercial success of current closure devices, certain obstacles may still exist in the event of device failure or other complications. For example, if the anchor was to dislodge from the plug prematurely, it may migrate downstream in the vasculature where it could damage the lower leg or cause other undesirable complications.

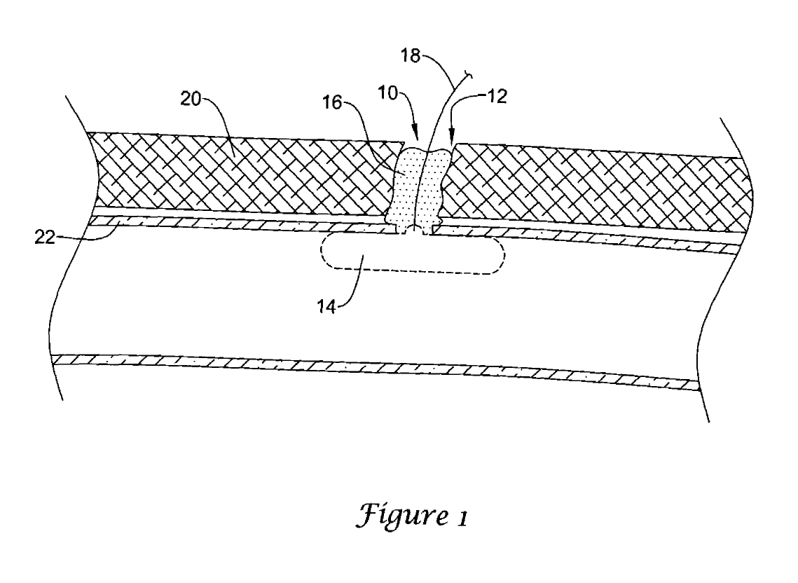

FIG. 1 illustrates a new closure device 10 disposed in a body opening 12 that may overcome some of the limitations of other closure devices. Closure device 10 may include an anchor 14, a plug 16, and a suture 18 attaching anchor 14 to plug 16. Device 10 is shown situated in body opening 12, which in this example is an opening through the skin 20 and into the femoral artery 22. As shown, anchor 14 may be configured so that at least a portion thereof is disposed within the femoral artery 22. Plug 16, conversely, may have at least a portion thereof disposed along the exterior of femoral artery 22. In at least some embodiments, plug 16 is configured so that it ends just below the skin 20. In other embodiments, plug 16 may terminate at the skin 20 exterior or at locations above the skin 20. It should be noted that although closure device 10 is shown closing an opening 12 into the femoral artery 22, this is not intended to be limiting as closure device 10 may be used to close essentially any suitable opening into a body lumen.

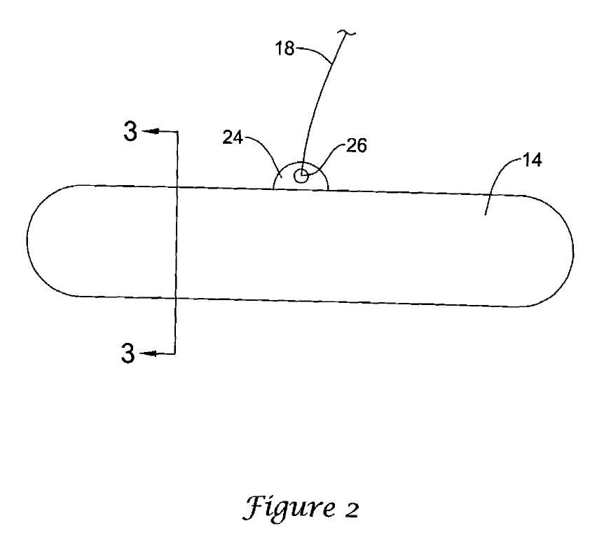

To facilitate the attachment of anchor 14 to plug 16, anchor 14 may also include a projection 24 having a suture opening 26 for suture 18 to extend therethrough as shown in FIG. 2. This may allow suture 18 to be arranged in a manner similar to a pulley or slip knot so that suture 18 can be pulled taught and secure anchor 14 together with plug 16. Some additional discussion regarding the attachment of anchor 14 to plug 16 can be found below.

Plug 16 may comprise any suitable material such as collagen. Any other suitable materials may be used including, for example, clot-promoting materials without departing from the spirit of the invention.

Unlike conventional anchors, anchor 14 may be designed so that is may be “rapidly dissolving”. This feature is emphasized in FIG. 1 in that anchor 14 is drawn in phantom line in order to indicate that anchor 14 may be dissolved or nearly dissolved a relatively short period of time after placing anchor. For the purposes of this disclosure, rapidly dissolving may be understood to mean that anchor 14 may be configured to dissolve more quickly within a body lumen than the 60-90 day time frame that may be typically observed in the art. Furthermore, rapidly dissolving may be understood to mean that anchor 14 may be configured to dissolve within a body lumen within about 30 days or less, or within about 24 hours or less, or within about 8 hours or less, or within 3 hours or less, or within about 5 minutes or less.

A number of design considerations may be utilized to form anchor 14 with desired rate of dissolution. For example, anchor 14 may be made from materials that dissolve relatively quickly in a body lumen (e.g., and/or when exposed to a biological environment). Suitable materials may include sugars (e.g., a monosaccharides, disaccharides, polysaccharide, etc.), polyanhidrides, polyesters (including, for example, polyglycolic acid, polylactide glycolide, etc.), starches, proteins, and the like, combinations thereof, or any other suitable material. In some embodiments, anchor 14 may include a polysaccharide formed into a glassy state.

The time period for these materials to dissolve within a body lumen or rate of dissolution may be generally known based on the properties of the material. For example, sugars may dissolve within about 1 to 30 minutes in a body lumen, or within about 1 to 10 minutes. Polyanhidrides may dissolve at times on the order of about 1 day or so. Polyesters may dissolve in about 30 days. The dissolutions times may also vary depending on thickness. For example, materials that are used as a coating or relatively thin layer may dissolve in a shorter time period. In addition, other additives may also be added to promote or slow the dissolution. For example, hydrogels or similar materials may be added to promote dissolution.

In addition to choosing materials based on their desired dissolution rate, anchor 14 may also include materials that are sufficiently strong and/or resistant to deformation so that can maintain their form while be sutured to plug 16 with suture 18. This may include the use of higher strength and/or slower dissolving materials adjacent to, for example, suture opening 26 so as to maintain the integrity of the bond between plug 16 and anchor 14 for the desired time period.

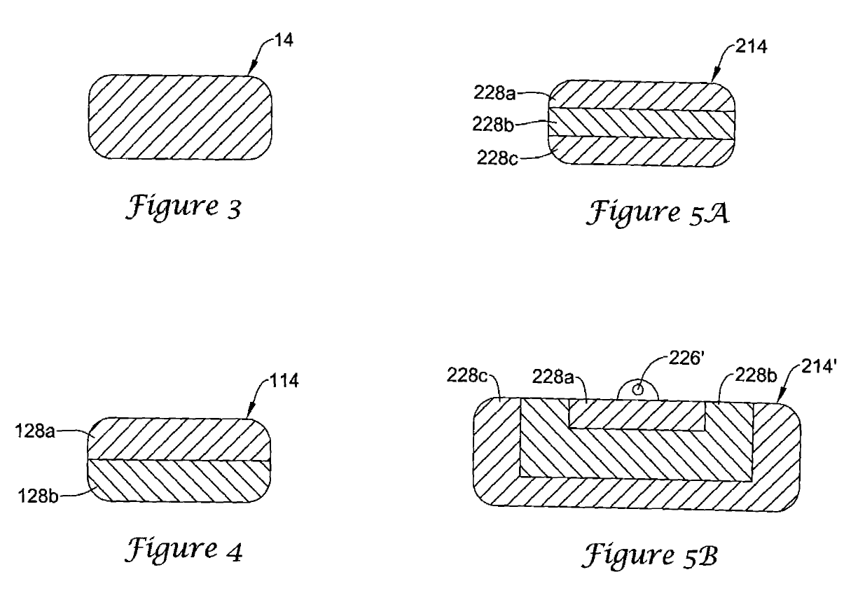

In at least some embodiments, anchor 14 may be single layered or otherwise be made from a single monolith of material as shown in FIG. 3. In other embodiments, anchor 14 may include a plurality (e.g., 1, 2, 3, 4, 5, 6, or more) of layers. For example, FIG. 4 illustrates another example anchor 114, which may be otherwise similar in form and function to any of the other anchors disclosed herein, that includes a first layer 128a and a second layer 128b. Similarly, FIG. 5A illustrates another example anchor 214, which may be otherwise similar in form and function to any of the other anchors disclosed herein, that includes a first layer 228a, a second layer 228b, and a third layer 228c. Of course, additional anchors are also contemplated that include more than three layers.

In some embodiments, the layers defined in any of the aforementioned anchors 114/214 may be distributed in a substantially uniform manner so that the layers extend longitudinally alongside each other. This need not be the case, however. For example, FIG. 5B illustrates anchor 214′ where layer 228b′ is disposed along a portion of the exterior of layer 228a′ and where layer 228c′ is disposed along a portion of the exterior of layer 228b′. Utilizing this configuration may allow the various layers of anchor 214′ to dissolve directionally toward suture opening 226′.

Because anchor 214′ is shown in a cross-sectional view that is taken transversely to the cross-section taken in earlier figures (including FIG. 5A), FIG. 5B may represent the configuration of layers utilized in anchor 214 or, analogously, other anchors. In the embodiment shown in FIG. 5B, it can be appreciated that layers 228a′/228b′/228c′ lay on top of the exterior of one another, yet do not fully envelop a more inwardly disposed layer. This, however, is not intended to be limiting as other anchors are contemplated were subsequent layers fully encapsulate more inwardly disposed layers.

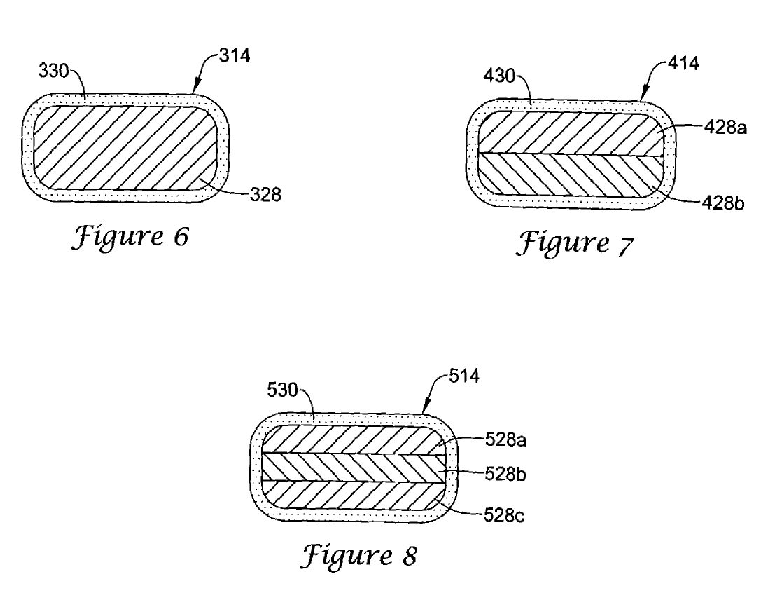

FIGS. 6-8 illustrate additional example anchors that may be similar in form and function to other anchors disclosed herein. However, the anchors illustrated in these figures include a relatively thin outer coating or layer. For example, FIG. 6 illustrates anchor 314 that includes a layer of material 328 coated with coating 330. Similarly, FIG. 7 illustrates anchor 414 that includes layers 428a/428b and coating 430. Likewise FIG. 8 illustrates anchor 514 that includes layers 528a/528b/528c and coating 530. Of course additional anchors are also contemplated that include different numbers of layers and/or one or more additional coatings.

The coatings described above may any of the materials disclosed herein or any other suitable material. For example, the coatings may include a poly(glycolic lactic) acid family of materials or other degradable polyesters. In some embodiments, the coating may include a material that dissolves at a somewhat slower rate than the layer(s) of material is covers. However, by virtue of it being thin, it may ultimately dissolve only somewhat slower, at the same rate, or even faster than the underlying layers.

In the embodiments of anchors that include different numbers and/or configurations of layers and/or coatings may utilize different materials for at least some of the layers and/or coatings. These materials may include materials that dissolve at different rates within a body lumen. Combining these materials may allow these anchors to have the desired rate of dissolution within the body lumen. For example, the anchors disclosed and contemplated may utilize two or more different materials, three or more different materials, four or more different materials, five or more different materials, six or more different materials, etc. Some embodiments of anchors may have a plurality of layers and/or coatings that are made from the same material. These embodiments may also include additional layers and/or coatings that are made from different materials.

With the above general configurations of layers and/or coatings in mind, any of the anchors disclosed above may be configured so as to have the desired dissolution time (and/or rate). For example, embodiments of anchors that include a single layer may include a material that has the desired dissolution time. Alternatively, a singular material having a generally high rate of dissolution may be mixed with materials that are less soluble. This may provide the mixture with the desired rapid dissolution time but at a rate that is longer than the monolithic material alone. For example, an example anchor may include a sugar mixed with protein(s), starch(es), and the like, combinations thereof, or any other suitable material.

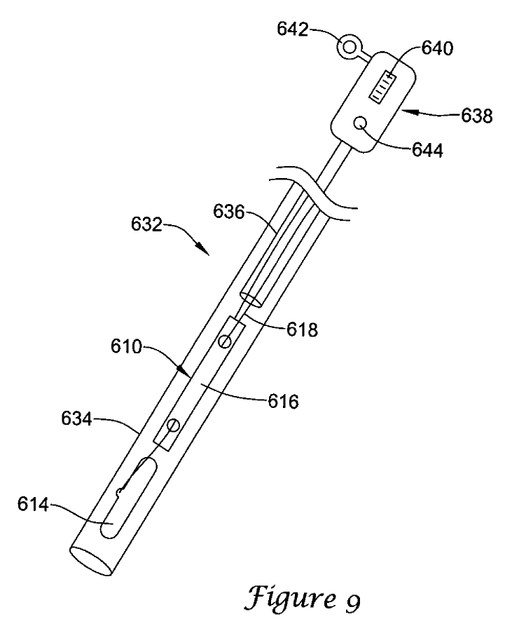

FIG. 9 illustrates a system 632 for delivering a closure device 610. Closure device 610 may be similar in form and function to any of the closures devices and/or components thereof as disclosed and contemplated herein. For example, closure device 610 may include anchor 614, plug 616, and suture 618. System 632 may include a sheath 634 for holding device 610. A push member 636 may be disposed in sheath 634, for example adjacent plug 616. System 610 may also include a handle 638. Handle 638 may include one or more control members such as a slider 640 (which may be coupled to and help control the position of anchor 614), an actuation cord or member 642 (which may retract sheath 634 when pulled as well as cinch and/or knot suture 618), and a visual indicator 644 (which may indicate forces present in system 632). Handle 638 may also include a number of different and/or alternative structural features.

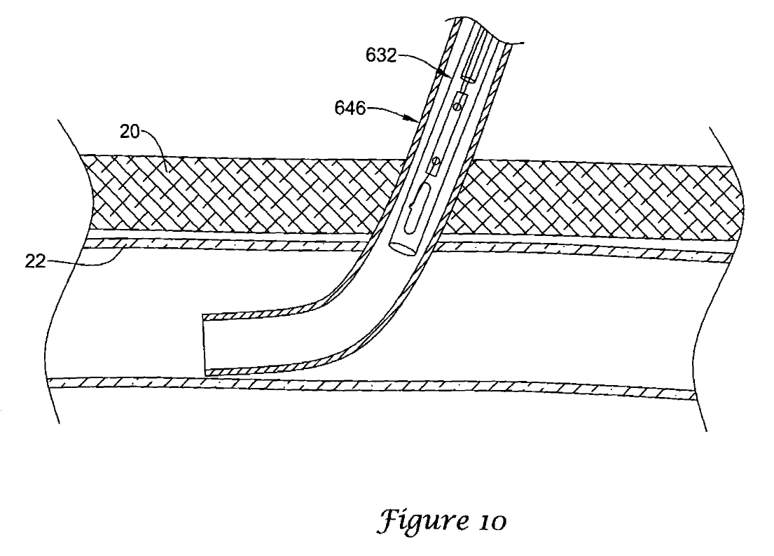

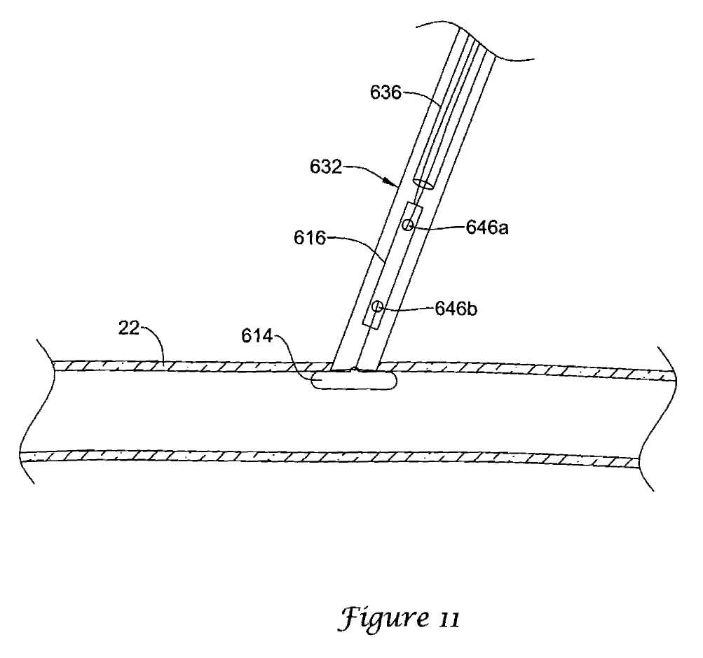

FIG. 10 illustrates system 632 disposed within an introducer sheath 646 extending through the skin 20 and into the femoral artery 22. This indicates that some embodiments of system 632 may be utilized with conventional introducer sheaths like sheath 646. This may be desirable because it allows system 632 to be utilized without the need of additional tools and/or structures. Alternatively, system 632 may be placed after removing introducer sheath 646. This may include the use of a typical obturator and/or dilator.

System 632 may be advanced through sheath 646 to a position where closure device 610 may be advanced out from sheath 634 and into the femoral artery 22. It can be seen in FIG. 10 that anchor 614 may be disposed so that its longer axis is aligned with the longitudinal axis of sheath 634. After emerging out from sheath 634, anchor 614 may be configured to shift or tilt so that the longer axis of anchor 614 is generally perpendicular to the longitudinal axis of sheath 634. Tilting may be accomplished in a number of different ways. For example, suture 618 may be configured or otherwise be arranged in a pulley-like manner with anchor 614 so that suture 618 can be pulled on in order to cause anchor 614 to tilt. The pulley-like arrangement may include one or more windings of suture 618 about portions of anchor 614 including the suture opening and/or projection (e.g., see projection 24 and opening 26 in FIG. 2 for reference) as may be utilized in the art.

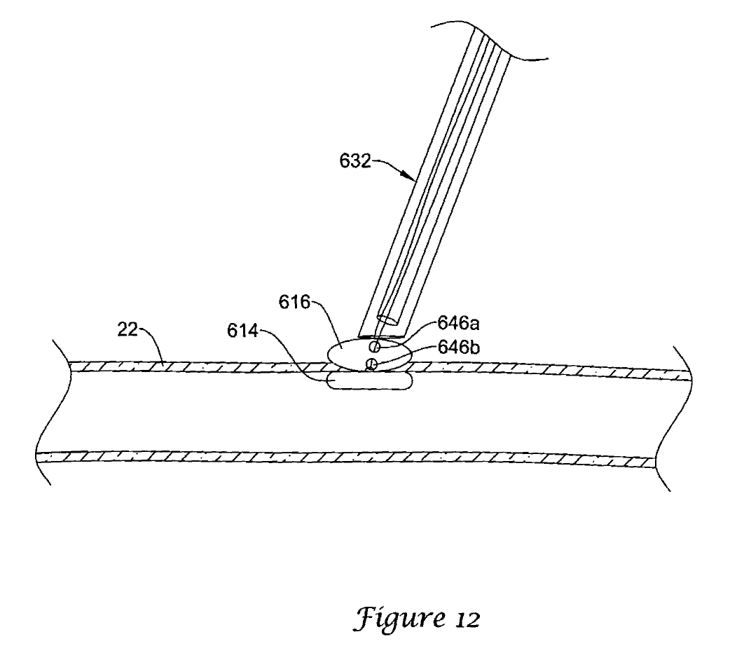

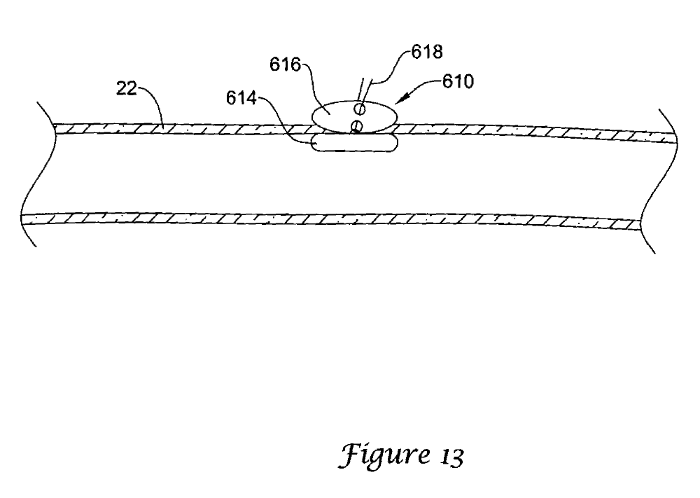

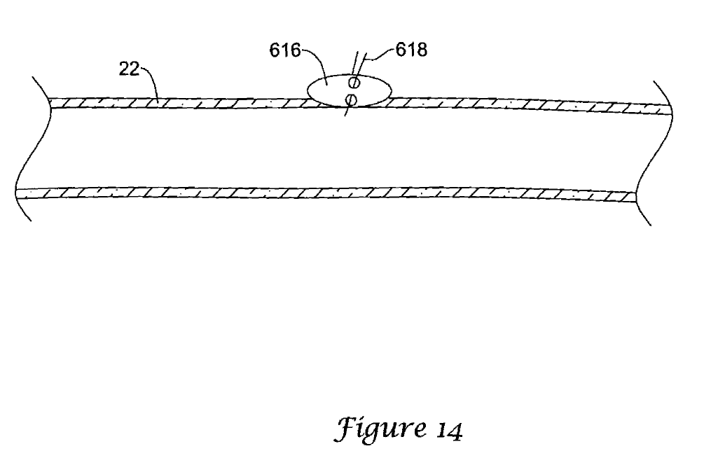

With anchor 614 properly arranged, sheath 634 and/or introducer 646 may be withdrawn proximally from the femoral artery 22 so that anchor 614 is positioned in the desired location. FIG. 11 illustrates system 632 backed into a position where anchor 614 abut the opening in femoral artery 22. For simplicity purposes, the skin 20 is not shown in this figure nor is introducer sheath 646, which the later may or may not be removed at this point in the procedure. At this point, push rod 636 may be distally advanced so as to push down on and deform plug 616 as shown in FIG. 12. In doing so, force may also be applied to suture 618, which may be threaded through anchor 614 as well as a plurality of openings (e.g., openings 646a/646b) in plug 616. This may tend to pull together and secure anchor 614 with plug 616. Such securing may be facilitated by a pulley-like arrangement between suture 618 and anchor 614. Once suitably configured, system 632 can be retracted, leaving behind closure device 610 (with suture 618 shown trimmed, for example, just below the skin line) as shown in FIG. 13. Finally, because anchor 614 may be rapidly dissolving, anchor 614 may dissolve, leaving behind plug 616 as shown in FIG. 14.

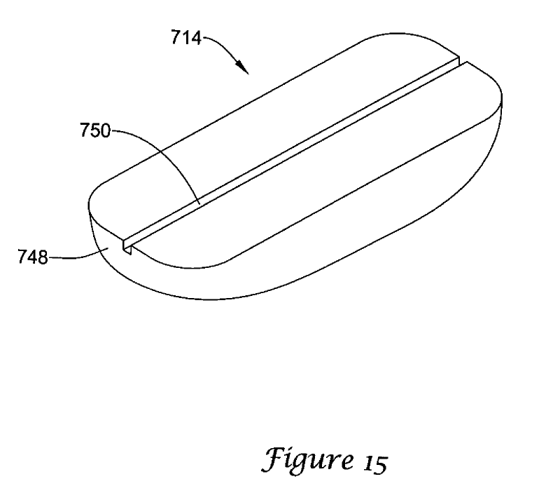

FIG. 15 illustrates another example anchor 714 that may be similar in form and function to other anchors disclosed herein. For example, anchor 714 may be rapidly dissolving. In addition, anchor 714 may include beveled edges 748 and a furrow or channel 750 along the middle thereof. Anchor 714, by virtue of bearing this configuration, may be foldable within a sheath (e.g., sheath 634), which may simply delivery of anchor 714 to its target location. For example, anchor 714 may be able to fold into a lower profile so that it can fit more easily within delivery and/or introducer sheaths.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A closure device for closing an opening in a body lumen, the closure device comprising:
a plug, wherein at least a portion of the plug is disposed adjacent an exterior surface of the body lumen;
a rapidly dissolving anchor, wherein the rapidly dissolving anchor is configured to dissolve within the body lumen within about 30 days or less and wherein at least a portion of the rapidly dissolving anchor is disposed within the body lumen; and
a suture coupling the plug to the anchor.

2. The closure device of claim 1, wherein the plug includes collagen.

3. The closure device of claim 1, wherein the rapidly dissolving anchor includes a sugar.

4. The closure device of claim 3, wherein the rapidly dissolving anchor includes a monosaccharide.

5. The closure device of claim 3, wherein the rapidly dissolving anchor includes a disaccharide.

6. The closure device of claim 3, wherein the rapidly dissolving anchor includes a polysaccharide.

7. The closure device of claim 1, wherein the rapidly dissolving anchor includes a polyanhidride.

8. The closure device of claim 1, wherein the rapidly dissolving anchor includes a polyester.

9. The closure device of claim 1, wherein the rapidly dissolving anchor includes a starch.

10. The closure device of claim 1, wherein the rapidly dissolving anchor includes a protein.

11. The closure device of claim 1, wherein the rapidly dissolving anchor includes a plurality of layers.

12. The closure device of claim 11, wherein the rapidly dissolving anchor includes two layers.

13. The closure device of claim 11, wherein the rapidly dissolving anchor includes three layers.

14. The closure device of claim 1, wherein the rapidly dissolving anchor includes four layers.

15. The closure device of claim 11 wherein at least one of the plurality of layers includes a thin outer coating.

16. The closure device of claim 11, wherein at least some of the plurality of layers are made from different materials.

17. The closure device of claim 16, wherein the plurality of layers are made from at least two different materials.

18. The closure device of claim 16, wherein the plurality of layers are made from at least three different materials.

19. The closure device of claim 16, wherein the plurality of layers are made from at least four different materials.

20. The closure device of claim 11, wherein at least some of the plurality of layers include a plurality of materials that dissolve within the body lumen at different rates.

21. The closure device of claim 1, wherein the rapidly dissolving anchor is configured to dissolve within the body lumen within about 24 hours or less.

22. The closure device of claim 1, wherein the rapidly dissolving anchor is configured to dissolve within the body lumen within about 8 hours or less.

23. The closure device of claim 1, wherein the rapidly dissolving anchor is configured to dissolve within the body lumen within about 3 hours or less.

24. The closure device of claim 1, wherein the rapidly dissolving anchor is configured to dissolve within the body lumen within about 5 minutes or less.

25. A system for delivering a closure device to an opening in a body lumen to close the opening, the system comprising:
a sheath;
a closure device disposed in the sheath, the closure device including a plug, a rapidly dissolving anchor, and a suture coupling the plug to the anchor;
wherein the rapidly dissolving anchor is configured to dissolve within the body lumen within about 24 hours or less; and
a push member disposed in the sheath adjacent the closure device, the push member being configured to advance the closure device out from the sheath.

26. The system of claim 25, further comprising an actuating handle for moving the push member.

27. The system of claim 25, further comprising means for moving the push member.

28. The system of claim 25, further comprising an introducer, wherein the sheath is disposed within the introducer.

29. The system of claim 25, wherein the plug includes collagen.

30. The system of claim 25, wherein the rapidly dissolving anchor includes a sugar, a polyanhidride, a polyester, a starch, a protein, or combinations thereof.

31. The system of claim 25, wherein the rapidly dissolving anchor includes a plurality of layers.

32. The system of claim 31, wherein at least one of the plurality of layers includes a thin outer coating.

33. The system of claim 31, wherein at least some of the plurality of layers are made from different materials.

34. The system of claim 3 1, wherein at least some of the plurality of layers include a plurality of materials that dissolve within the body lumen at different rates.

35. The system of claim 25, wherein the rapidly dissolving anchor is configured to dissolve within the body lumen within about 24 hours or less.

36. The system of claim 25, wherein the rapidly dissolving anchor is configured to dissolve within the body lumen within about 8 hours or less.

37. The system of claim 25, wherein the rapidly dissolving anchor is configured to dissolve within the body lumen within about 3 hours or less.

38. The system of claim 25, wherein the rapidly dissolving anchor is configured to dissolve within the body lumen within about 5 minutes or less.

39. A method for closing an opening in a body lumen, the method comprising the steps of:
providing a closure device delivery system, the system comprising:
a sheath,
a closure device disposed in the sheath, the closure device including a plug, a rapidly dissolving anchor, and a suture coupling the plug to the anchor,
wherein the rapidly dissolving anchor is configured to dissolve within the body lumen within about 24 hours or less, and
a push member disposed in the sheath adjacent the closure device, the
push member being configured to advance the closure device out from the sheath;
disposing a portion of the sheath within the body lumen;
advancing the closure device out from the sheath with the push member;
closing the opening in the body lumen with the closure device; and
removing the sheath and the push member from the body lumen.
